(19) United States
(12) Patent Application Publication
(10) Pub. No.: US 2021/0353406 A1
(43) Pub. Date: Nov. 18, 2021

(54) Accommodative Functionality for Artificial Capsule Platform

(71) Applicants: The Board of Trustees of the Leland Stanford Junior University, Stanford, CA (US); The Regents of the University of California, Oakland, CA (US)

(72) Inventors: Frank L. BRODIE, Durham, NC (US); Stephen D. MCLEOD, San Francisco, CA (US)

(21) Appl. No.: 17/284,561

(22) PCT Filed: Oct. 22, 2019
(86) PCT No.: PCT/US19/57503

§ 371 Date: Apr. 12, 2021

Related U.S. Application Data
(60) Provisional application No. 62/749,262, filed on Oct. 23, 2018.
(60) Provisional application No. 62/806,772, filed on Feb. 16, 2019.
(60) Provisional application No. 62/851,202, filed on May 22, 2019.

(51) Int. Cl. A61F 2/16

(52) U.S. Cl. CPC A61F 2/1629; A61F 2210/0014; A61F 2002/169; A61F 2220/0008; A61F 2002/16902

(57) ABSTRACT

A method of using an implantable artificial intraocular lens capsule to provide accommodative functionality through forward backward movement. The implantable artificial capsule has a ring with a central opening to accommodate an intraocular lens and at least three haptic arms extending from the ring. Visual accommodation is accomplished by forward or backward motion of the artificial intraocular lens which is made possible by the flexibility or stimuli induced shape change of the haptic arms. Stimuli include (i) mechanical forces acting on the haptic arms for example by the ciliary muscle, (ii) gravitational forces acting on the artificial intraocular lens, (iii) forces acting indirectly on the haptics arms or (iv) external forces including electrical or electromagnetic delivered via the externalized haptics.

### FIELD OF THE INVENTION

This invention relates to device, method and system of using an artificial capsule platform to provide pseudo-accommodative and accommodative function.

### BACKGROUND OF THE INVENTION

Accommodation of eye sight is achieved by changing the focal length with the eye to bring near objects into focus. In young people this is achieved by changing the shape of the natural lens to increase the dioptric power due to ciliary muscle contraction. Ciliary muscle contraction occurs anytime someone attempts near focus even if they are already presbyopic or lost accommodation due to hardening of the natural lens.

Presbyopia, or loss of ocular accommodative function, occurs either with natural age-related changes of the lens or in the case that the natural lens is replaced with an artificial intraocular lens. Regardless of the etiology, patients lose the functional ability to dynamically change their focal point. While the natural lens changes its dioptric power by becoming more convex to achieve a near focal point for close work, a similar functional outcome can be achieved by moving a lens of a fixed dioptric power forward within the eye or by changing the refractive index or surface properties of the lens. Moving the lens forward induces a myopic shift allowing for near objects to be viewed clearly—with the focal point depending on the dioptric power of the lens, the axial length of the eye and the movement of the lens. The present invention provides technology by which this type of accommodation can be achieved by means of an implanted intraocular artificial lens capsule. It also provides a direct connection of an intraocular device through the wall of the eye to a protected subconjunctival space for direct signal delivery to an intraocular device.

### SUMMARY OF THE INVENTION

Loss of accommodation with age or cataract surgery greatly impacts patients' quality of life. The loss of clarity to read and do detail work at distances less than 1 meter leads to great frustration and numerous imperfect solutions are adopted such as reading glasses, monovision or intraocular lenses with multifocal optics. An ideal solution with replicated the dynamic focus of the natural lens. The movement of the lens capsule complex in response to stimuli, or accommodative signal transduction through the wall of the eye to an intraocular device which can then change optical property and focus power as described herein, provides this ideal type of solution. This approach utilizes all available light to a single desired focal point for maximum clarity. This approach allows users to enjoy focus at the full range of focal lengths without compromise of clarity. The focal length can be dynamically titrated to the activity. Various internal or external stimuli can be used to facilitate ease of use and optimize response time and precision.

In one embodiment a method of using an implantable artificial intraocular lens capsule is provided to enable accommodative functionality and dynamically change a focal length. The implantable artificial intraocular lens capsule has a ring with a central opening to accommodate an intraocular lens and at least three haptic arms extending from the outer surface of the ring and attached to an eye wall (e.g. slcera, pars plana, pars plicata, ciliary body and/or iris).

Using this embodiment, the haptic arms allow forward or backward motion of the intraocular lens when a person wearing the implantable artificial intraocular lens capsule moves its head respectively forward or backward due to gravity changes acting on the intraocular lens.

Using this same embodiment, the haptic arms could also allow forward or backward motion of the intraocular lens when a person wearing the implantable artificial intraocular lens capsule changes contraction of one or more of the ciliary muscles causing a transfer of forces, directly or indirectly, via the one or more of the at least three haptic arms onto the intraocular lens.

In a variation of this embodiment, a second set of haptic arms could be added which are different from the at least three haptic arms. The second set of haptic arms are located within or pass through the ciliary muscles. Using this embodiment, the second set of haptic arms allow forward or backward motion of the intraocular lens when a person wearing the implantable artificial intraocular lens capsule changes contraction of the ciliary muscle causing transfer of forces via the one or more of the at least three haptic arms on to the intraocular lens.

In another embodiment, a method of using an implantable artificial intraocular lens capsule is provided to enable accommodative functionality and dynamically change a focal length. The implantable artificial intraocular lens capsule has a ring with a central opening to accommodate an intraocular lens and at least three haptic arms extending from the outer surface of the ring and attached to an eye wall. The haptic arms allow flexing in a forward or a backward motion of the intraocular lens when a person wearing the implantable artificial intraocular lens capsule in response to an electro-magnetic stimulus either under voluntary or automatic control of the person.

In this embodiment, the haptics could be made of a material that has an inherent shape change in response to the electro-magnetic stimulus. Furthermore, the haptics arms could include or made out if a shape-metal alloy, an electroactive polymer or the like.

### BRIEF DESCRIPTION OF THE DRAWINGS

FIG. 1 shows the artificial capsule according to exemplary embodiments of the invention.

FIGS. 2-5 show the artificial capsule and the physical implementation of the artificial capsule according to a first exemplary embodiment of the invention. The first exemplary embodiment pertains to an artificial capsule with a central opening that has an inward facing groove to accommodate intraocular lens haptics or other intraocular implants.

FIGS. 6-10 show the artificial capsule and the physical implementation of the artificial capsule according to a second exemplary embodiment of the invention. The second exemplary embodiment pertains to an artificial capsule that has a flat design with a central opening to accommodate intraocular lens haptics or other intraocular implants.

FIGS. 11-13 show examples of accommodative functionality of the artificial capsule according to embodiments of the invention.

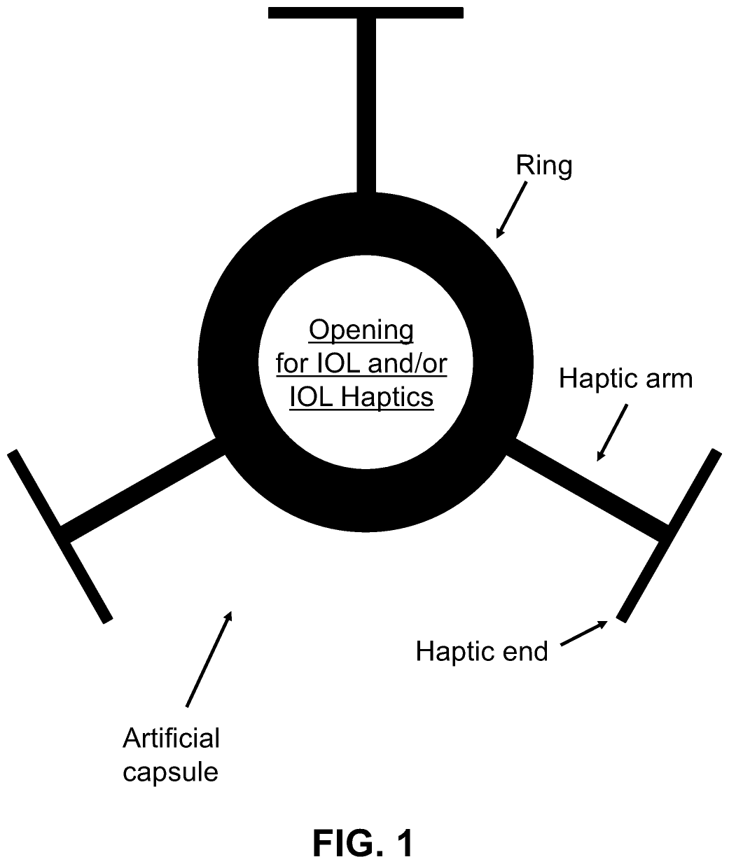

### DETAILED DESCRIPTION

### Intraocular Artificial Lens Capsule

The present invention utilizes an artificial capsule with suture-less scleral fixation which would recapitulate normal anatomy. Scleral fixation allows external communication of the intraocular device across the wall of the eye allowing direct communication across the sclera both for signal and for power, including electrical. The artificial capsule has a central ring to support the intraocular lens while, in one embodiment, 3 haptics (arms) would be externalized trans-sclerally and sit subconjunctivally, ensuring a secure position while remaining covered to prevent erosion or infection. Sutureless placement would be rapid and technically straightforward. The artificial capsule would be made of a flexible biocompatible material such as silicon, polymethylmethacrylate or acrylic (both common IOL materials currently used). Other features of suitable materials are biocompatible, opaque or transparent. It could be inserted through a standard corneal incision and the haptics would be externalized through small gauge sclerotomy incisions. The capsule would accommodate a variety of common IOLs. The artificial capsule allows for the implantation of the IOL at the same time as placement, or IOL insertion could be delayed until a later date and only require a short anterior segment procedure for insertion.

### Applications of Intraocular Artificial Lens Capsule

Embodiments of the invention have applications in cataract surgery when there is absent or severely compromised capsular support. This occurs in cases of trauma, surgical complication or with disease processes such as pseudoexfoliation or marfans syndrome. As described herein, embodiments of the intraocular artificial lens capsule could also have application in the accommodative functionality of vision.

The artificial capsule is intended to provide support and centration for an intraocular lens (IOL) without use of the native capsular bag. It is intended to be used in cases of deficient capsular and/or zonular support, either congenital or secondary to disease, trauma or iatrogenic injury. The artificial capsule can hold the lens in the sulcus plane or in the posterior chamber. The artificial capsule haptics can sit a-traumatically within the eye or one or more haptics can be externalized for scleral fixation. The IOL can be placed within the artificial capsule either at the time of implantation or at a later date. The IOL can be exchanged without removal of the artificial capsule.

Additionally, the lens-capsule complex can provide a flexible platform that allows for anterior-posterior movement in response to external or internal stimuli. As a result of this movement accommodative functionality will be achieved. Stimuli could include, but are not limited to, gravity from eye tilt/rotation, mechanical forces externally applied including in response to ciliary body contraction and electro-magnetic forces either externally or internally driven. The communication of this device with the artificial capsule which bridges the sclera to be externalized in the subtenons or subconjunctival space allows a direct route for signals to control initiation and termination of accommodation, as well as extraocular powering of electrical devices.

### Design Examples of Intraocular Artificial Lens Capsule

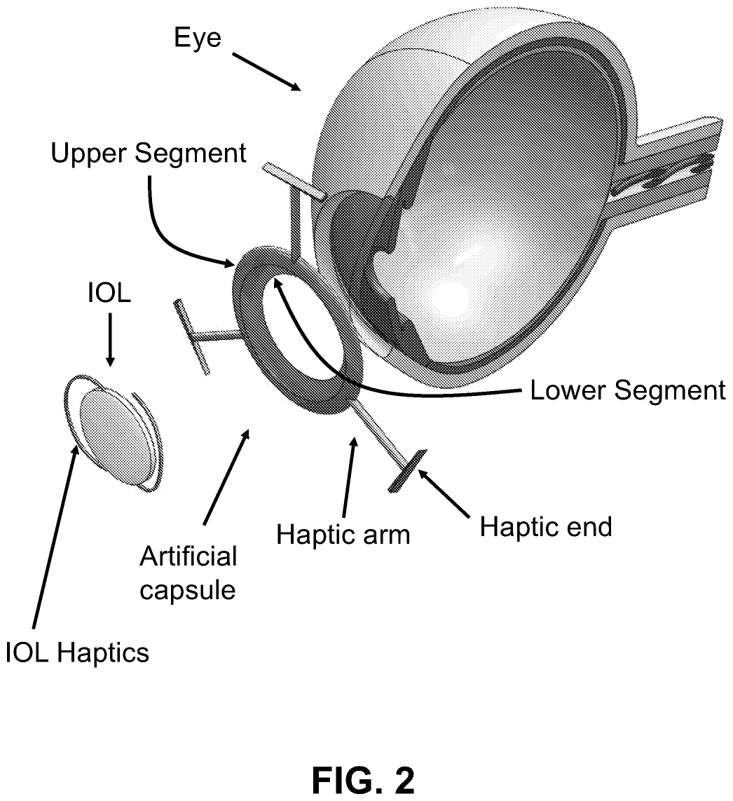

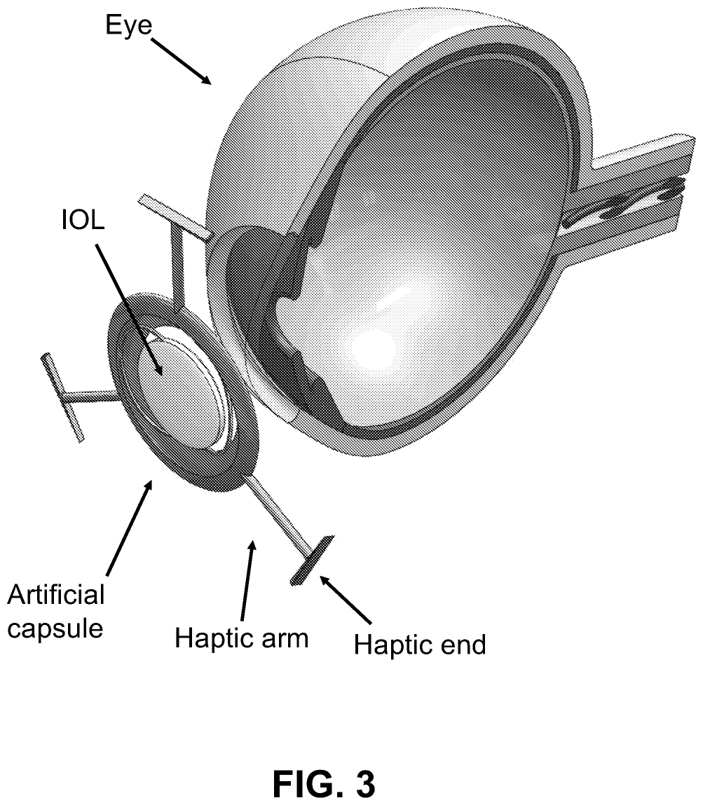

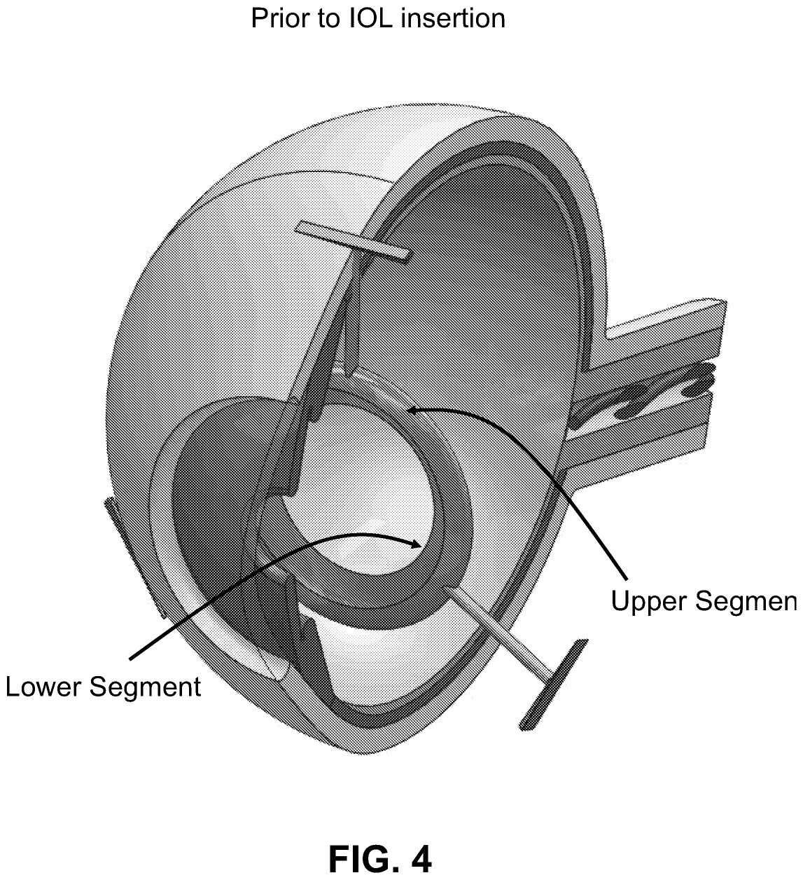

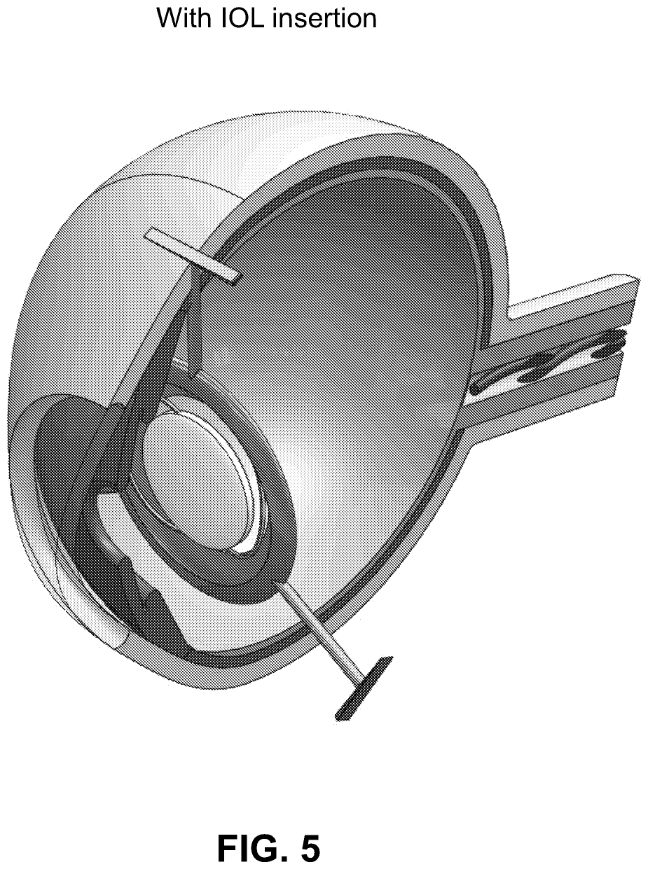

In a first example, the artificial capsule is a single object with two major components (FIGS. 2-5):
1. Circular ring with a central inward facing groove to accommodate the IOL and/or IOL haptics.
2. Capsular haptics coming out of the ring which can fasten to the sclera or sclerotomies.

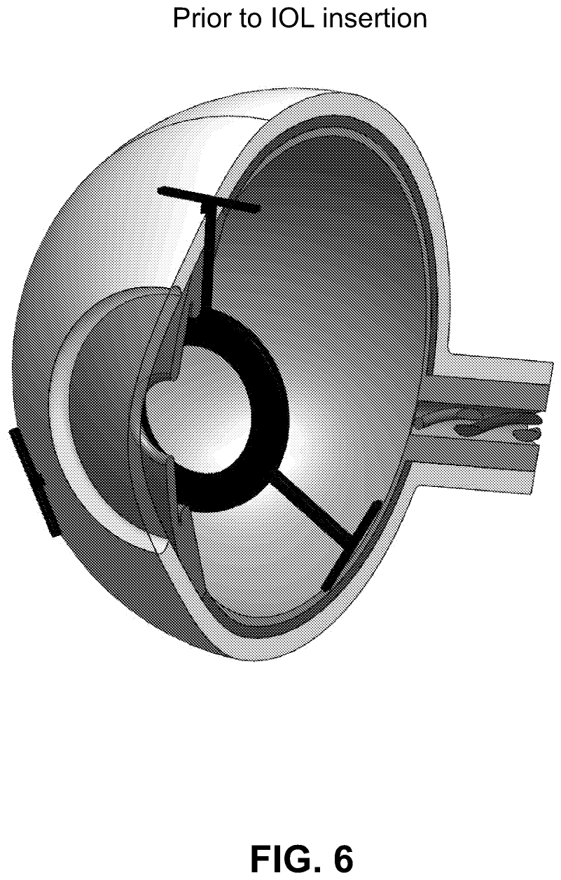

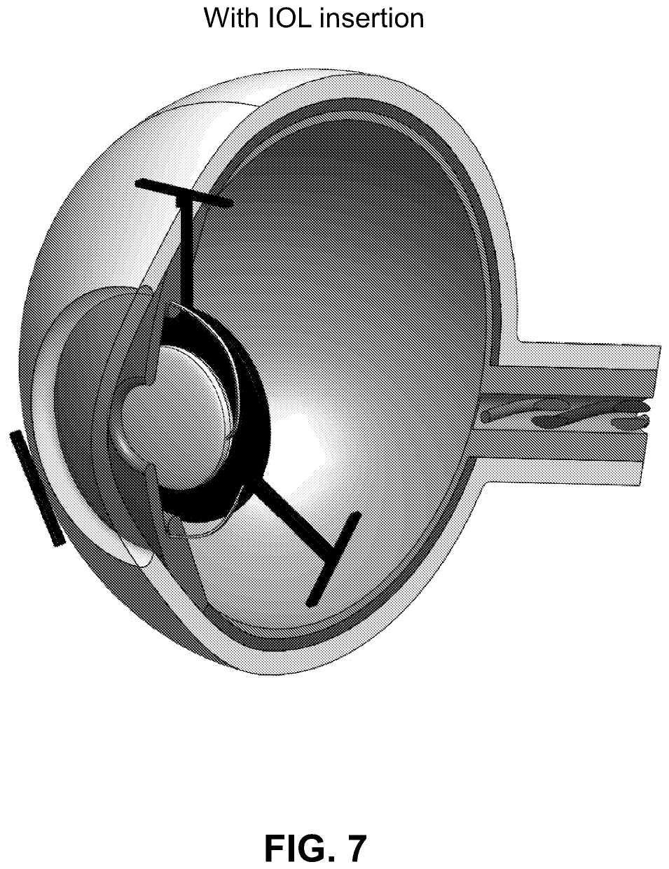

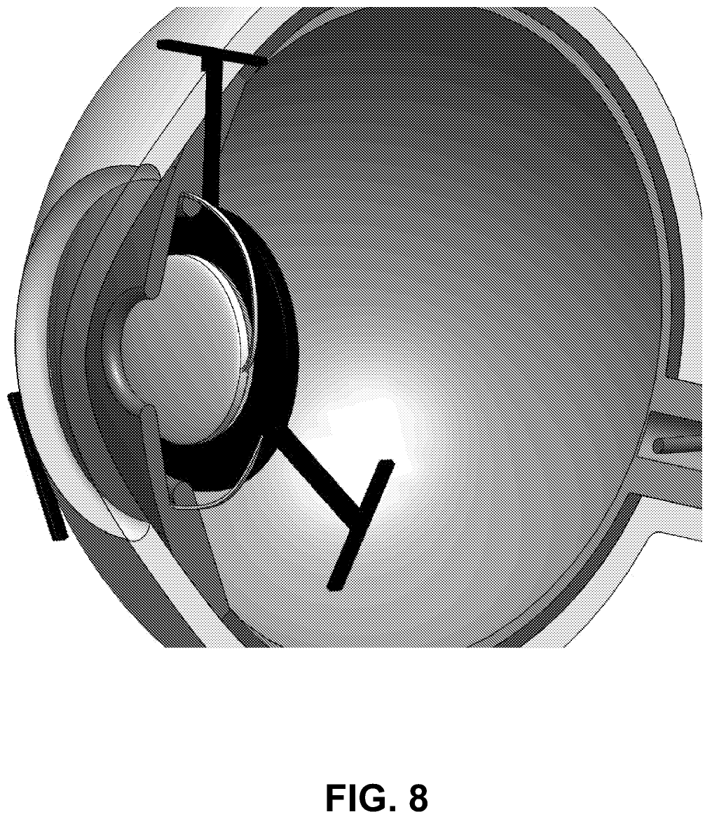

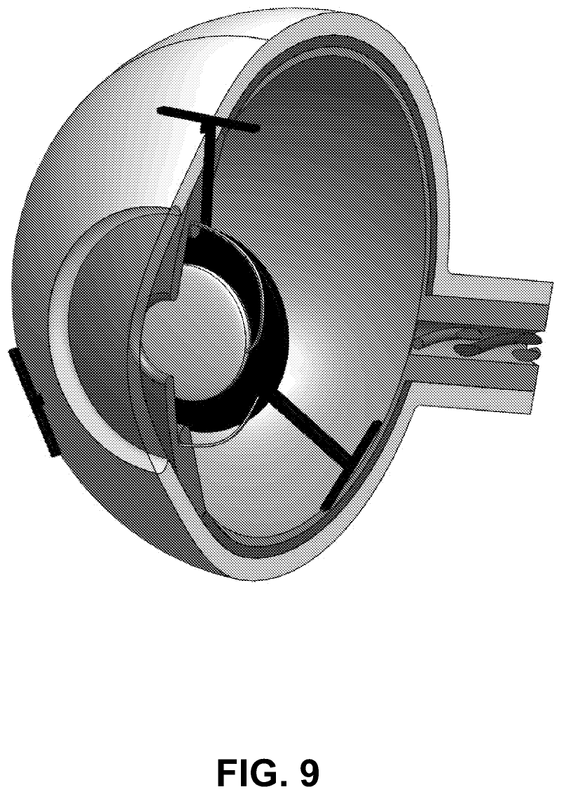

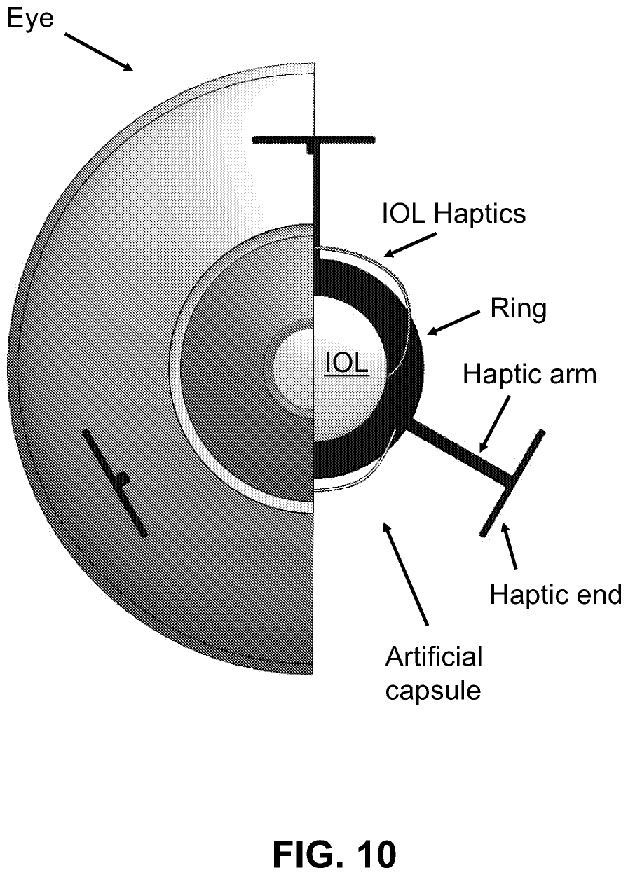

In a second example, the artificial capsule is a single object with two major components (FIGS. 6-10):
1. Flat circular ring where the flat ring part is designed to support IOL and/or IOL haptics. 2. Haptics coming out of the ring which can fasten to the sclera or
sclerotomies.

### Accommodative Functionality of Intraocular Artificial Lens Capsule

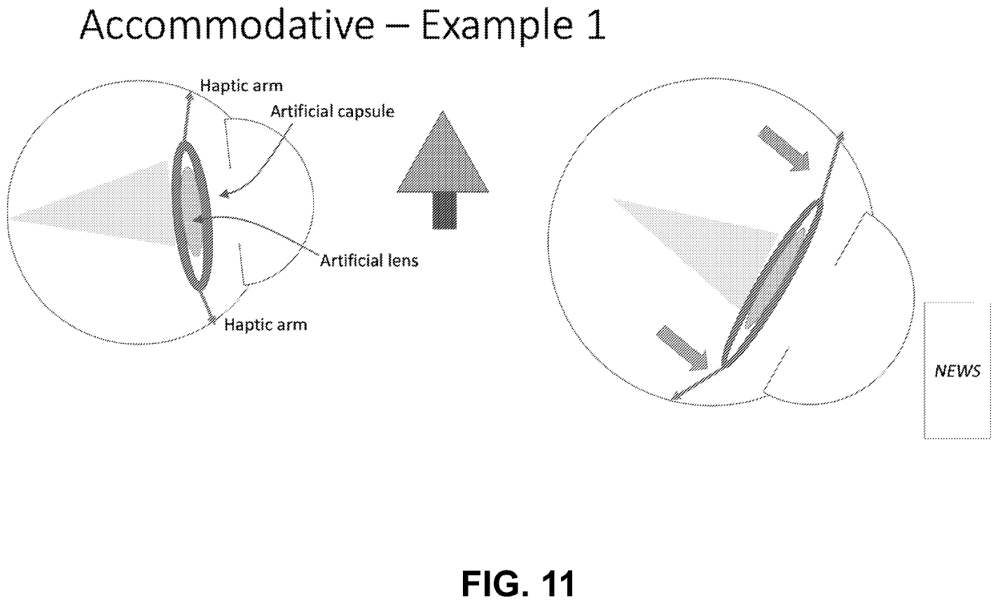

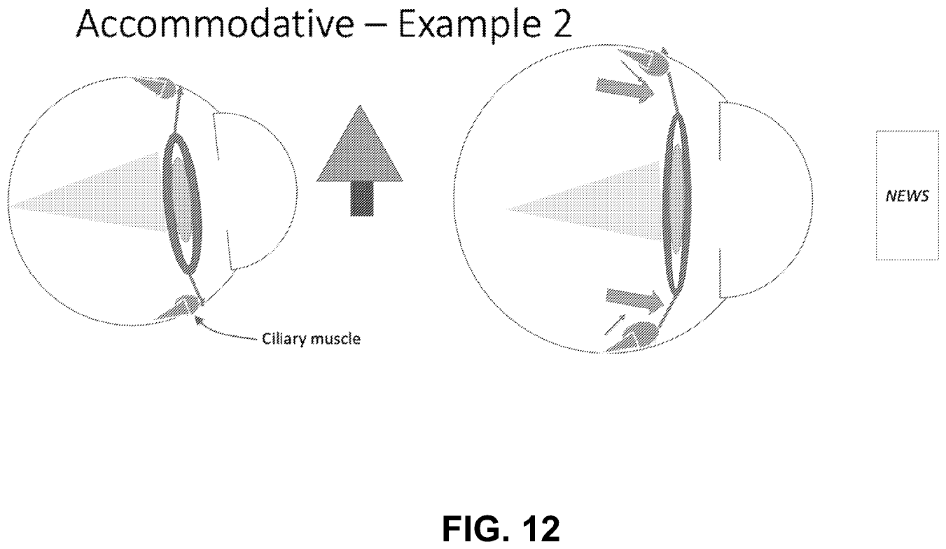

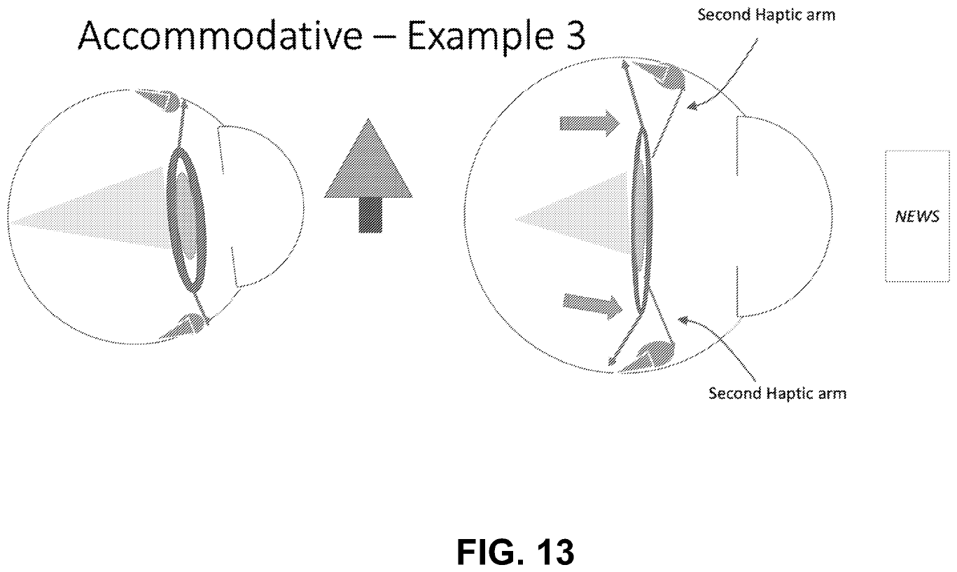

The inventors of this invention discovered that if certain flexibility is added and provided to the haptic arms of the intraocular artificial lens capsule it will allow the intraocular artificial lens to shift forward and backward within the eye while the intraocular lens (IOL) remains secure. The forward and backward movement effectively provides accommodative function. The direction and magnitude of the motion can be achieved with (see also FIGS. 11-13): rotation of the eye in which gravity drives motion of the capsule-lens complex, mechanical movement possibly linked to ciliary body movement or other intra- or extra-ocular forces, or an electromagnetic stimulus for movement.
1) FIG. 11 shows an example of accommodation where gravity is utilized and given the flexibility of the haptic arms when the user looks down to read, the platform shifts forward and the focal point moves forward, providing near vision. In other words, the shift forward occurs with downgaze and gravity pulls the artificial lens implant forward. This is reversed as the patient looks up again and the lens-capsule complex falls back, restoring emmetropia.
2) FIG. 12 shows an example where accommodation takes place in response to ciliary muscle contraction (this is a component of the physiologic near response). In this embodiment that contraction is either directly or indirectly coupled with anterior movement of the lens-capsule complex—the resultant myopic shift provides a near focal point allowing for pseudo-accommodation. Subsequent ciliary body relaxation allows the lens-capsule complex to move posteriorly to the emmetropic position.
3) FIG. 13 shows an example where a second set of haptics (note that these are different from the haptic arms needed for anchoring the implant) can engage the ciliary muscle to respond to accommodation. This second set of haptics can come off the artificial capsule platform or the IOL. In other words, the shift forward with the ciliary muscle shape change results in the mechanical force translated by the second set of haptics directly engaging the ciliary muscles.
4) In yet another example, not shown, the forward/backward motion of the artificial lens implant can be stimulated to an external force, such as electrical or magnetic stimulation or another mechanical force.

1. A method of using an implantable artificial intraocular lens capsule to provide accommodative functionality and dynamically change a focal length, wherein the implantable artificial intraocular lens capsule comprises a ring with a central opening to accommodate an intraocular lens and at least three haptic arms extending from the outer surface of the ring and attached to an eye wall, and wherein the haptic arms:
(i) allow forward or backward motion of the intraocular lens when a person wearing the implantable artificial intraocular lens capsule moves its head respectively forward or backward due to gravity changes acting on the intraocular lens, or
(ii) allow forward or backward motion of the intraocular lens when a person wearing the implantable artificial intraocular lens capsule changes contraction of one or more of the ciliary muscles causing a transfer of forces, directly or indirectly, via the one or more of the at least three haptic arms onto the intraocular lens.

2. The method as set forth in claim 1, wherein the eye wall comprises a slcera, a pars plana, a pars plicata, a ciliary body or an iris.

3. The method as set forth in claim 1, further comprising a second set of haptic arms, different from the at least three haptic arms, wherein the second set of haptic arms are located within or pass through the ciliary muscles, and wherein the second set of haptic arms allow forward or backward motion of the intraocular lens when a person wearing the implantable artificial intraocular lens capsule changes contraction of the ciliary muscle causing transfer of forces via the one or more of the at least three haptic arms on to the intraocular lens.

4. A method of using an implantable artificial intraocular lens capsule to provide accommodative functionality and dynamically change a focal length, wherein the implantable artificial intraocular lens capsule comprises a ring with a central opening to accommodate an intraocular lens and at least three haptic arms extending from the outer surface of the ring and attached to an eye wall, and wherein the haptic arms allow flexing in a forward or a backward motion of the intraocular lens when a person wearing the implantable artificial intraocular lens capsule in response to an electro-magnetic stimulus either under voluntary or automatic control of the person.

5. The method as set forth in claim 4, where the haptics are made of a material that has an inherent shape change in response to the electro-magnetic stimulus.

6. The method as set forth in claim 4, wherein the haptics arms comprise a shape-metal alloy or an electroactive polymer.
